(19) United States
(12) Patent Application Publication
(10) Pub. No.: US 2008/0109078 A1
(43) Pub. Date: May 8, 2008

(54) POSTERIOR CHAMBER PHAKIC INTRAOCULAR LENS

(75) Inventors: George W. Rozakis, North Olmsted, OH (US); Igor G. Valyunin, Hobooken, NJ (US); Alexander P. Hatsis, Massapequa, NY (US)

Correspondence Address: FULBRIGHT & JAWORSKI, LLP, 1301 MCKINNEY, SUITE 5100, HOUSTON, TX, 77010-3095, US

(73) Assignee: Implantable Vision, Inc., New York, NY (US)

(21) Appl. No.: 11/875,402

(22) Filed: Oct. 19, 2007

Related U.S. Application Data
(60) Provisional application No. 60/853,100, filed on Oct. 20, 2006.

(51) Int. Cl. A61F 2/16

(52) U.S. Cl. 623/6.43

(57) ABSTRACT

The invention relates to a novel phakic intraocular lens. The positioning arms or haptics of the lens are designed to hold the lens in position and proper orientation without engaging structures within the eye.

### CROSS-REFERENCE TO RELATED APPLICATIONS

This Application is a Non-Provisional of Provisional (35 USC 119(e)) application 60/853,100 filed on Oct. 20, 2006.

### STATEMENT REGARDING FEDERALLY SPONSORED RESEARCH OR DEVELOPMENT

Not applicable.

### REFERENCE TO A COMPACT DISK APPENDIX

Not applicable.

### TECHNICAL FIELD

This invention generally relates to an intraocular lens and, more particularly, to a posterior chamber, phakic intraocular lens. One configuration of the present invention is directed to a phakic intraocular lens having a lens positioning arm having a platform. An exemplary lens having the platform includes three positioning arms.

### BACKGROUND OF THE INVENTION

Various posterior chamber, phakic intraocular lenses are known in the art. These lenses are implanted directly behind the iris in front of the eye's natural lens. One drawback with these lenses is the need for an iridotomy that allows fluid to flow from the posterior chamber to the anterior chamber of the eye. The art desires an implant that may be used without an iridotomy. Another drawback with known lenses is the limitation on the size of the optical portion of the lens. The art desires a lens with a large optical portion. The art also desires a lens having a configuration that does not interfere with the fluid flow patterns in the eye while having a structure that maintains a desired location within the eye. Typical known lenses use haptics that span the eye chamber and engage opposed portions of the ciliary bodies to wedge the lens in place. Other lenses use the iris to create centering forces on the lens. The art desires a phakic lens that does not relay on as much contact with the eye to remain in a desired position as known lenses.

The advantages of these lenses are that the flat front surface of the lens can have a larger diameter than lenses with curved front surfaces. The large diameter and large radius of the posterior optical surface allow the lens to be formed in a wide range of optical powers such as those that are needed by patients who are ineligible for corneal laser surgery. The large diameter optical portion also minimizes halos. The large flat surface minimizes pressure on the iris so as to avoid iris chafing. Further, the channels of the invention allow fluid flow even when the joint of the lens contacts the iris. The lens may thus be implanted without an iridotomy. The thick rim disposed about the optical portion of the lens maintains the lens in the desired location.

There remains, however, a need for an improved phakic intraocular lens which provides improved positioning stability.

### BRIEF SUMMARY OF THE INVENTION

The present invention relates to an intraocular lens with improved positioning stability.

In one configuration, the invention provides a phakic intraocular lens having a flat front surface and a curved rear optical surface to define the optical power of the lens. The lens may be used with or without an iridotomy. The lens has positioning arms that help maintain the position of the lens within the eye. Different configurations for the positioning arms are disclosed. In one configuration, the invention provides a platformed positioning arm that allows more aqueous to be disposed behind the lens adjacent the anterior surface of the crystalline lens. The platformed positioning arm may be incorporated into two arm lens designs and three arm lens designs.

In a further configuration, the invention provides a three-positioning arm lens design for a posterior chamber, phakic intraocular lens. The three-positioning arm lens is designed to be easy to insert behind the iris. The positioning arms are configured to allows the lens to float behind the iris in front of the crystalline lens without the need to vault the lens or fixate the ends of the positioning arms. On configuration of the three-arm lens is configured to maintain a predictable position within the eye.

Another aspect of the invention is the method of designing the lens based on the measurements of the eye.

When properly sized and implanted in the eye, different lens configurations of the invention will accommodate when the zonular fibers engage the ends of the positioning arms to drive the optical body forward or cause the positioning arms to flex the optical body.

### BRIEF DESCRIPTION OF THE DRAWINGS

FIG. 1 is a sectional view of the eye having a phakic intraocular lens implanted next to the natural lens;

FIG. 2 is a top plan view of an alternative lens configuration having two platformed positioning arms or haptics;

FIG. 3 is a section view taken along line 12-12 of FIG. 2;

FIG. 4 is a section view taken along line 13-13 of FIG. 2;

FIG. 5 is a top plan view of another lens configuration;

FIG. 6 is a section view taken along line 17-17 of FIG. 5;

FIG. 7 is an enlarged section view of the end of the positioning arm of FIG. 5;

FIG. 8 is a top plan view of another lens configuration;

FIG. 9 is a section view of an arm of the lens in FIG. 8;

FIG. 10 is an enlarged section view of the end of the positioning arm of FIG. 9;

FIG. 11 is a top plan view of another lens configuration having three positioning arms;

FIG. 12 is an end view of the lens shown in FIG. 11;

FIG. 13 shows various arm configurations.

Similar numbers refer to similar elements throughout the specification.

### DETAILED DESCRIPTION OF THE INVENTION

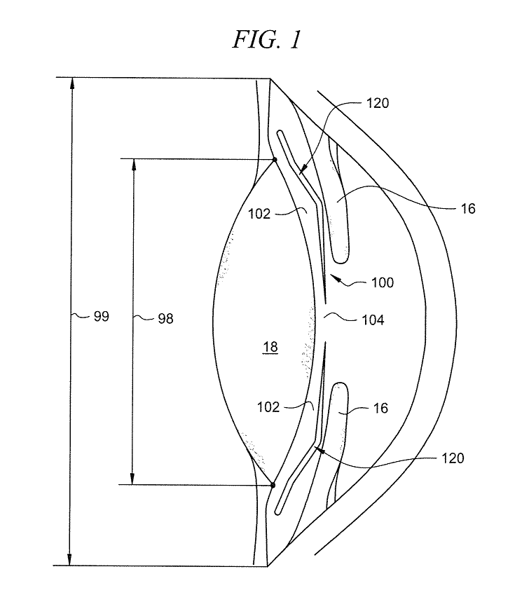

The lens described below may be implanted in the eye by folding the lens and slipping the folded lens through the pupil of the eye. As shown in FIG. 1, once implanted in the posterior chamber of the eye, lens 100 provides accommodation when the outer ends of positioning arms 120 are manipulated by the ciliary body or the zonular fibers that connect lens 18 to the ciliary body of the eye. The ciliary body and zonular fibers expand into the posterior chamber of the eye when eye accommodates. Lens 100 is able to take advantage of this movement by having the floating outer ends of arms 120 closely positioned adjacent the ciliary body and zonular fibers when lens 100 is floating. During accommodation, the ciliary body and/or zonular fibers engage arms 120 and push lens 100 toward iris 16 causing accommodation. The ciliary body and/or zonular fibers may also force arms 120 toward each other (radially inwardly) to flex the optical body of lens 100 thus decreasing radius 36. Arms or haptics 120 are relatively stiff compared with many prior art haptics so that the arms 120 effectively transmit the force of the zonular fibers to the optical portion of lens 100 because of the relatively thick rim 41 and the cupped configuration of arms 120 and the body of the lens 100. The ends of the haptics or arms should have a radius of curvature equal to the radius of curvature of the ciliary suculus. This helps ensure that the haptics do not contain any of the internal ocular structures.

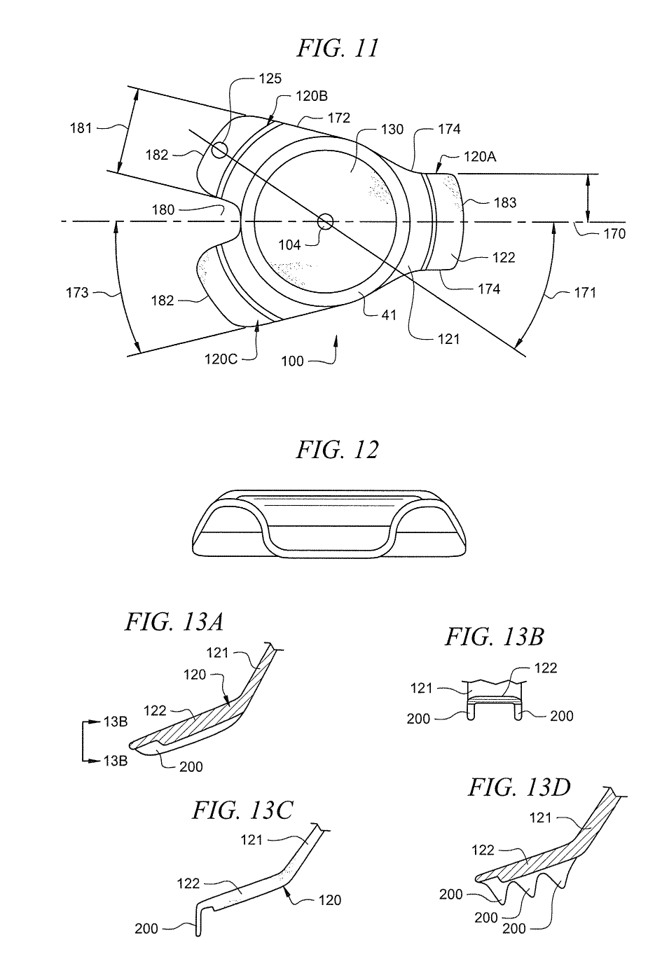

A lens is indicated generally by the numeral 100 in FIGS. 11 and 12. Lens 100 is designed to be a posterior chamber, phakic intraocular lens that may be inserted behind iris 16 but in front of lens 18. Lens 100 includes at least a pair of two-part positioning arms 120 but may also include three (as shown in FIG. 3) or more two-part positioning arms 120. Each positioning arm 120 is platformed to provide a lens configuration that may be designed with a large optical radius 36 while also providing a relatively deep pocket 102 that receives aqueous from the eye. Platformed arms 120 and pocket 102 allow a larger volume of aqueous to be disposed between the anterior surface of lens 18 and the posterior surface of lens 100. When lens 100 is formed with an opening 104 at the optical portion of lens 100, the fluid of the eye flows behind arms 120 along the anterior surface of lens 18 and through opening 104. This allows the flow of aqueous between anterior and posterior chambers using adequate flow of nutrients to the lesser in the two regions. It also eliminates the need for an iridotomy.

Platformed arms or haptics 120 also allow the lens designer to position the ends of arms 120 close to the ciliary body and zonular fibers so that lens 100 will accommodate when the body and fibers engage arms 120. This positioning may be accomplished because platformed arms 120 allow the depth of lens to be increased without increasing the overall diameter of the lens to a degree that would wedge the lens within the eye. Lens 100 may thus remain floating within the eye.

One method of sizing the overall outer diameter is to measure (such as with ultrasound) the outer diameter 98 of lens 18 and the outer diameter of posterior chamber 99. The outer diameter of lens may be designed to be half of the sum of these two measurements. Such an outer diameter allows lens 100 to float within the eye after implantation as long as the depth of lens 100 is designed to prevent lens 100 from becoming wedged between the crystalline lens 18 and the iris 16. The ultrasound measurements may be used to define this depth and the angles of the arms 120 described below.

In one embodiment of the invention, lens 100 is customer manufactured for a patient by measuring the eye and the posterior chamber and then cutting lens 100 from a material (such as by a lathe and a material such as acrylic) instead of molding lens 100. Cutting lens 100 provides for an efficient manner of manufacturing lens for a particular patient. Once the lens is manufactured, the lens may be heated to a temperature that matches the eye before implantation. Heating helps the lens be folded for implantation and helps the lens to unfold once implanted. Another method is to load the lens into a sterile injection cartridge before it is shipped to the surgeon. This method prevents the surgeon from loading the lens in the injector. This method, however, requires the lens to be manufactured form a material that allows the lens to immediate regain its desired shaped after implantation.

To aid the surgeon in positioning the lens after insertion, various structures can be created in the arms. For example, a small indentation may be placed in one arm of the lens. This allows the surgeon to insert a probe or similar instrument to the indentation and move the lens accordingly. Alternatively, a revised structure can be used. The shape of the structures can vary and includes, but is not limited to, squares, circles, crescents and the like.

In one embodiment, a central operation 104 is provided which allows fluid communication from the anterior to the posterior portions of the lens and between the anterior and posterior chambers. The presence of the application permits the free flow of fluid between the two chambers, eliminating the need for an iridotomy.

In an alternate embodiment, ridges are cut into the outer edge of the lens rim again allowing free flow of fluid to both chambers.

Each two-part platformed arm or haptic 120 includes an inner arm portion 121 and an outer arm portion 122. The inner arm portion 121 integrally extends posteriorly and radially outwardly from rim 41 to the inner end of outer arm portion 122. Outer arm portion 122 extends posteriorly and radially outwardly from the outer end of inner arm portion 121. Outer arm portion 122 extends, however, at an angle that is more shallow with respect to the flat front surface 130 of the optical body of lens 100. Arm portion 122 is not, however, disposed parallel to anterior surface 130. Reference plane 133 is parallel to anterior surface 130 while reference plane 134 is parallel to anterior surface 135 (disposed along the same radius of lens 100 as shown in FIG. 3). Reference plane 136 is parallel to anterior surface 137. The acute angle between reference plane 133 and 134 falls in a broad range to allow the lens to fit to a variety of eye sizes. Angle 138 must be greater than the acute angle between plane 136 and plane 133 and may be greater than this angle by at least 10 degrees to define the platform of arm 120.

In one configuration, angle 138 may be between 75 degrees and 15 degrees and more specifically between 30 and 50 degrees. In one particular configuration, angle 138 is 45 degrees and angle 139 is 165 degrees. The acute angle between plane 136 and plane 133 should always be greater than 15 degrees.

The posterior surface of inner arm portion 121 may be substantially parallel to surface 135 such that arm portion 121 has a constant thickness. In other embodiments, the arm portion 121 may taper slightly down from rim 41 toward arm portion 122.

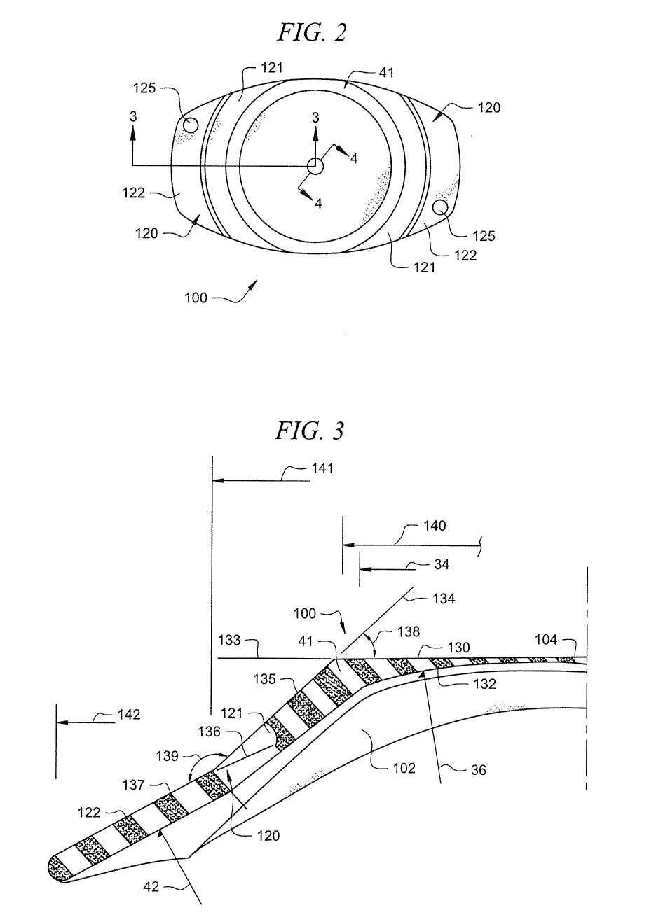

In the lens depicted in FIGS. 2 and 3, the anterior surface 130 of the optical body is flat as described above with the posterior surface 132 providing the optical curvature 36 of lens 100. The optical radius 36 may be in the range of 12 mm to 24 mm. The posterior surface of arm portion 122 may have a radius described above and may be 10 mm. The platformed arms 120 allow radius 36 to be increased without increasing the overall diameter of lens 100 thus allows the outer ends of arms 120 to be designed to be disposed radially outwardly of lens 18. The exemplary diameter 34 of the flat optical portion is 6 mm with an optical radius 36 of 13.43 mm. The center of the optical body defines the thinnest portion of the optical body which is limited by the material properties of lens 100. The center of the lens may optionally define an opening 104 to allow fluid to flow through lens 100. Opening 104 may have a diameter from about 0.1 mm to 0.6 mm. As also was described above, a rim 41 surrounds the outer periphery of the optical body. The platformed arms 120 decrease the thickness of rim 41 while maintaining the relative position of the anterior surface of rim 41 with the posterior surface of the outer ends of arms 120.

In one configuration, the transition between arm portions 121 and 122 has a diameter 141 of 9 mm with the overall diameter 142 of lens 100 being 11.3 mm. The outer diameter 140 of posterior surface 132 is 7 mm. Diameter 34 is 6 mm. Arm portions 122 are 0.2 mm thick. Radius 36 is 13.43 mm while radius 42 is 10 mm.

In another configuration having the same outer diameter 142 of 11.3 mm, transition diameter 141 is 9 mm while optical radius 36 is 23.43 mm. Diameter 34 is 5.5 mm. Arm portions 122 are 0.2 mm thick. Radius 42 is 10 mm.

These configurations are exemplary and the dimensions change based on such factors as the desired optical power of lens 100.

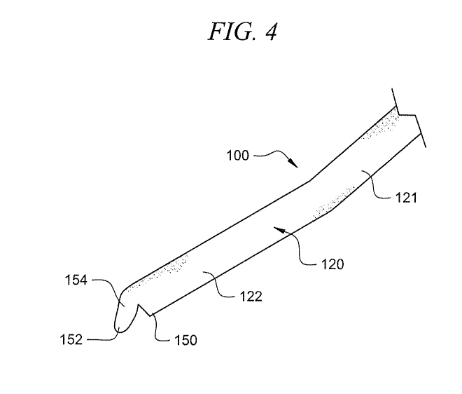

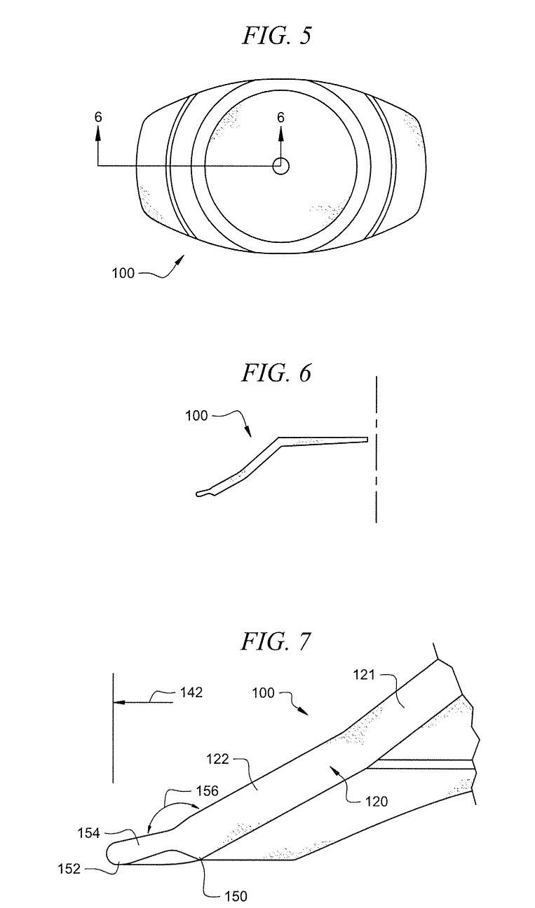

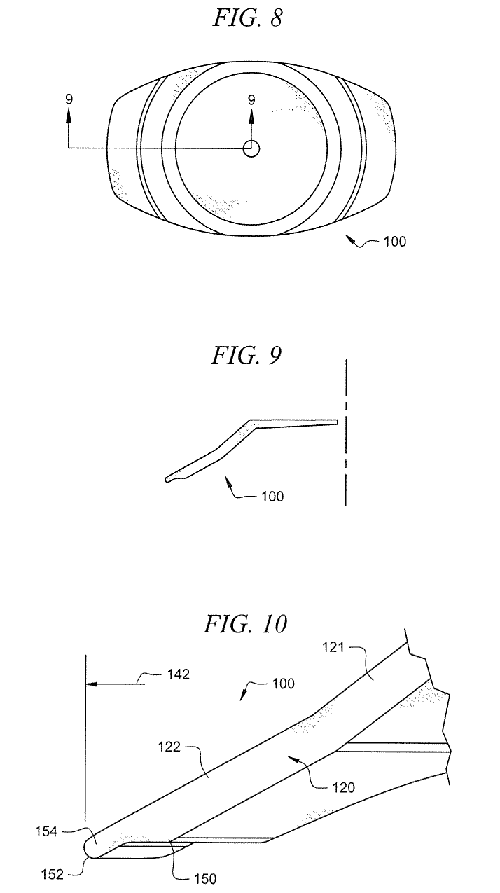

FIGS. 4-7 depict an alternative lens 100 wherein the ends of arm portions 122 are stepped to provide two spaced apart and distinct posterior ends 150 and 152 for lens 100. Ends 150 and 152 are created by extending a tip 154 from the outer end of arm portion 122. Tip 154 may have a thickness less than the thickness of arm portion 122 and may be on the order of 0.10 mm. End 152 is the point that is most posterior on lens 100. The posterior spacing between ends 150 and 152 is defined by the length of tip 154 and the angle which tip 154 is disposed with respect to arm portion 122. Angle 156 may be similar or less than angle 139 and may be about 170 degrees. Ends 150 and 152 provide two different locations on lens 100 for the eye to engage and move lens 100 after lens 100 is implanted. In this configuration, each arm 120 has three arm sections 121, 122, and tip 154 that are each disposed at a different angle with respect to anterior surface 130. In FIG. 19-21, angle 156 is 180 degrees thus spacing end 150 further anteriorly with respect to end 152 than in FIG. 18. These thin arm tips minimize contact between lens 100 and the eye or the structure supporting the eye while maintaining pockets 102.

FIG. 11 depicts a configuration for lens 100 wherein three positioning arms 120 are used to position lens 100 within the eye. This arrangement is sometimes called a tripod configuration. This lens may be designed to accommodate as described above. The three arm configuration may be used to relatively fix the orientation of lens 100 within the eye. In some situations, the eye is non-symmetric such that lens 100 may be implanted with arm 120A disposed aligned with the long dimension of the eye. This configuration will prevent lens 100 from freely rotating within eye 100 even though lens 100 is floating within the posterior chamber. In another embodiment, arms 120B and 120C may be made heavier to cause lens 100 to return to a configuration with arm 120A disposed upwardly. Arms 120B and 120C may be made heavier by making them twice as thick as arm 120A.

In one configuration, diameter 34 is 6 mm with diameter 142 being 11.5 mm. The optical radius 36 is 13.43 mm. Each arm 120 has one of the structures described above. The two arms 120B and 120C that are closest together are angled from centerline 170 by an angle 171 of 35 degrees while the other arm 120A is disposed on centerline 170. The outer sidewall 172 of each of these arms 120 is angled from the centerline at an angle 173 of 12.46 degrees. Walls 172 are tangent to rim 41 while the outer walls 174 of the center arm 120 are inset from tangent to reduce the size of center arm 120A. Reducing the size of centered arm 120A allows the mass of the center arm 120A to be reduced with respect to the combined masses of the offset arms 120B and 120C. Inset walls 174 also allow lens 100 to rolled or folded into the shape of a dart for easier insertion into the eye or an injector. The notch 180 defined between arms 120B and 120C has an inner end disposed at the thick rim 41 so that the injector plunger will push directly against the thick rim 41 when lens 100 is being injected into the eye. The size of notch 180 may be varied by varying the width 181 of arms 120B and 120C. Widths 181 may be varied so that the combined length of the tips 182 of arms 120B and 120C are equal to the length of the tip 183 of arm 120A. Widths 181 may also be varied to make the area of combined arms 120B and 120C equal to arm 120A.

Another manner of maintaining the position of a lens within an eye is to provide fingers 200 projecting posteriorly from the posterior surface of arm portion 122 as shown in FIG. 24. These fingers may interact with the zonular fibers to prevent the lens from freely rotating. Different configurations are depicted in FIG. 24.

Lens embodiments may be manufactured from a silicone material although some extremely thin members described herein may not be able to be manufactured from silicone. Any of the lens embodiments of the invention may be fabricated from an acrylic. A hydrophobic acrylic having a UV inhibitor and a blue blocker may be used. The material may have an index of refraction of 1.499 and allows portions of the lens to be formed as thin as 40 microns. However, various lens materials are known in the art. For instance, it is know that the optical portions of intraocular lenses may be fabricated from polymethyl methacrylate, poly-2-hydroxyethyl methacrylate, methyl methacrylate copolymers, siloxanylalkyl, fluoroalkyl and aryl methacrylate, silicone, silicone elastomers, polysulfones, polyvinyl alcohols, polyethylene oxides, copolymers of fluoroacrylates and methacrylate, and polymers and copolymers of hydroxyalkyl methacrylate, such as 2-hydroxyethyl methacrylate, as well as methacrylic acid, acrylic acid, acrylamide methacrylamide, N,N-dimethylacrylamide, and N-vinylpryrrolidone. Additionally, compounds that absorb ultraviolet or other short wavelength (e.g. below about 400 nm) radiation, such compounds derived from benzotriazole groups, benzophenone groups, or mixtures thereof may be added to the monomers and/or polymers that constitute the implant.

In the foregoing description, certain terms have been used for brevity, clearness, and understanding. No unnecessary limitations are to be implied therefrom beyond the requirement of the prior art because such terms are used for descriptive purposes and are intended to be broadly construed.

Moreover, the description and illustration of the invention is an example and the invention is not limited to the exact details shown or described.

What is claimed is:

1. A phakic intraocular lens having a central optical body and at least a pair of positioning arms extending radially outwardly from the outer periphery of the optical body; the positing arms comprising:
an inner arm portion connected to the optical body; and
an outer arm portion connected to the inner arm portion; the outer arm portion being disposed at a more shallow angle with respect to the optical portion than the inner arm portion.

2. The arm of claim 1, wherein the outer tip of the outer arm portion defines a pair of spaced posterior tips.

3. The arm of claim 1, wherein the outer tip of the outer arm portion defines a tripod configuration.

4. The arm of claim 1, further comprising a ridge projecting from the anterior surface of the outer arm portion.

5. The arm of claim 1, further comprising a ridge projecting from the anterior surface of the inner arm portion.

6. The arm of claim 1 wherein the edge of the arm has a radius of curvature equal to the radius of curvature of the ciliary suculus of a patient's eye.

7. A phakic intraocular lens disposed in an eye; the configuration comprising:
a phakic lens having an optical portion and a pair of platformed positioning arms;
the posterior tips of the positioning arms being disposed adjacent the ciliary body or zonular fibers that support the crystalline lens of the eye;
the optical portion of the phakic lens being moved when the ciliary body or zonular fibers move to accommodate the crystalline lens.

8. The lens of claim 7, wherein the phakic lens define an opening at the optical portion of the phakic lens.

9. The lens of claim 8, wherein the platformed positioned arms define at least a pair of fluid pockets between the crystalline lens of the phakic lens.

10. The arm of claim 1 wherein the edge of the arm has a radius of curvature equal to the radius of curvature of the ciliary suclu of a patient's eye.
